(19) United States
(12) Patent Application Publication
(10) Pub. No.: US 2023/0284870 A1
(43) Pub. Date: Sep. 14, 2023

(54) COMPUTER VISION BASED CONTROL OF AN ENERGY GENERATOR

(71) Applicant: Covidien LP, Mansfield, MA (US)

(72) Inventors: Christopher T. Brown, Boulder, CO (US); Joe D. Sartor, Longmont, CO (US); Jing Zhao, Superior, CO (US); Thomas E. Drochner, Longmont, CO (US)

(21) Appl. No.: 18/019,881

(22) PCT Filed: Jul. 15, 2021
(86) PCT No.: PCT/US2021/041779

§ 371 Date: Feb. 6, 2023

Related U.S. Application Data
(60) Provisional application No. 63/062,700, filed on Aug. 7, 2020.

(51) Int. Cl. A61B 1/00; A61B 18/14; A61B 18/12

(52) U.S. Cl. CPC A61B 1/00006; A61B 18/1206; A61B 18/1445; A61B 2018/0063

(57) ABSTRACT

An energy-based surgical system includes at least one electrosurgical instrument and an endoscope configured to capture video data of a surgical site including the at least one energy instrument. The system also includes an endoscope controller configured to process the video data to determine contextual data pertaining to one of the surgical site or the at least one energy instrument. The system further includes an energy generator coupled to the at least one energy instrument, the energy generator configured to output energy to the at least one energy instrument and to control energy based on the contextual data.

### BACKGROUND

### Technical Field

The present disclosure relates to an energy-based surgical system having an energy generator that is controlled using machine vision, and in particular, identification of instrument type, state, and location.

### Background of Related Art

Electrosurgery involves application of high radio frequency (RF) electrical current to a surgical site to cut, ablate, desiccate, or coagulate tissue. In monopolar electrosurgery, a source or active electrode delivers radio frequency alternating current from the energy generator to the targeted tissue. A patient return electrode is placed remotely from the active electrode to conduct the current back to the generator.

In bipolar electrosurgery, return and active electrodes are placed in close proximity to each other such that an electrical circuit is formed between the two electrodes (e.g., in the case of an electrosurgical forceps). In this manner, the applied electrical current is limited to the body tissue positioned between the electrodes. Accordingly, bipolar electrosurgery generally involves the use of instruments where it is desired to achieve a focused delivery of electrosurgical energy between two electrodes. Electrosurgical instruments are also used in laparoscopic surgery, where the clinician has limited view due to the presence of a single viewpoint supplied by an endoscope. Ultrasonic instruments are also used which include ultrasonic transducers that vibrate waveguides and attached end effectors, e.g., sealers and/or dissectors, to seal or dissect tissue due to heat imparted by rapid movement of the end effectors. Ultrasound is controlled by closure of jaw and extent of vibration and multiple energy or lengths of tip displacement are used to seal or cut tissue between the jaws or cut tissue in contact with the end of the jaws. Other thermal devices less commonly used include lasers in direct contact or stand off from the tissue target designed to coagulate or ablate using either high or low energy pulses. Additionally, ferromagnetic or positive thermal coefficient heating of a tissue contacting element have been used to cut, coagulate, seal or thermally necrose.

Energy delivery may be controlled using algorithms, which utilize voltage and/or current based closed loop control schemes. Algorithms only control energy delivery and do not prevent activation of the instruments beyond the field of view (e.g., in laparoscopic surgery) or activation of the instrument in various misuse conditions such as clamping on large tissue bundles, or other situations which may result in inadvertent injury of tissues.

Thus, there is a need for novel automated control algorithms that utilize advancements in computer vision technology utilizing machine learning to add context to the electrosurgical control schemes.

### SUMMARY

According to one embodiment of the present disclosure, an energy-based surgical system is disclosed. The energy-based surgical system includes at least one energy instrument being either ultrasound, electrosurgical, ferromagnetic, laser or resistive heating and an endoscope configured to capture video data of a surgical site including the at least one energy instrument. The system also includes an endoscope controller configured to process the video data to determine contextual data pertaining to one of the surgical site or the at least one energy instrument. The system further includes an energy generator coupled to the at least one energy instrument, the energy generator configured to output energy to the at least one energy instrument and to control the energy based on the contextual data.

According to one aspect of the above embodiment, the contextual data includes at least one of type, operational state, position, or orientation of the at least one energy instrument. The at least one energy instrument is operable in a plurality of configurations. The energy generator is further configured to select an energy mode based on a configuration of the at least one energy instrument.

According to another aspect of the above embodiment, the endoscope controller is further configured to determine whether the at least one energy instrument is obstructed or outside of field of vision of the endoscope. The energy generator is further configured to disable the output of RF energy based on determination whether the at least one energy instrument is at least one obstructed or outside of field of vision of the endoscope.

According to a further aspect of the above embodiment, the at least one energy instrument is a bipolar forceps configured to form a tissue seal. The endoscope controller is further configured to determine at least one parameter of the tissue seal. The energy generator is configured to adjust the output of RF energy based on the at least one parameter of the tissue seal.

According to a further embodiment of the present disclosure, an energy-based surgical system is disclosed. The energy-based surgical system includes a plurality of electrosurgical instruments and an endoscope configured to capture video data of a surgical site including the electrosurgical instruments. The system also includes an endoscope controller configured to process the video data to determine contextual data pertaining to one of the surgical site or the energy instruments and to identify each of the plurality of energy instruments. The system further includes an energy generator coupled to the energy instruments, the energy generator configured to output radio frequency (RF) energy to the energy instruments and to control RF energy based on the contextual data.

According to one aspect of the above embodiment, the contextual data includes at least one of type, operational state, position, or orientation of each of the plurality of energy instruments. The endoscope controller is further configured to determine whether one of the plurality of energy instruments is obstructed or outside of field of vision of the endoscope and the energy generator is further configured to disable the output of RF energy based on determination whether one of the plurality of energy instruments is at least one obstructed or outside of field of vision of the endoscope.

According to a further embodiment of the present disclosure, a method for controlling an energy generator is disclosed. The method includes capturing video data of a surgical site and at least one energy instrument through an endoscope and processing the video data at an endoscope controller to determine contextual data pertaining to one of the surgical site or the at least one energy instrument. The method further includes outputting radio frequency (RF) energy to the at least one electrosurgical instrument from an energy generator and controlling RF energy based on the contextual data.

According to one aspect of the above embodiment, the method further includes operating the at least one electrosurgical instrument in one of a plurality of configurations. The method also includes selecting at the energy generator an electrosurgical mode based on a configuration of the at least one electrosurgical instrument. The method includes determining whether the at least one electrosurgical instrument is obstructed or outside of field of vision of the endoscope. The method also includes disabling the output of RF energy based on determination whether the at least one electrosurgical instrument is at least one obstructed or outside of field of vision of the endoscope.

According to another aspect of the above embodiment, the at least one electrosurgical instrument is a bipolar forceps configured to form a tissue seal. The method also includes determining at least one parameter of the tissue seal. The method further includes adjusting the output of RF energy based on the at least one parameter of the tissue seal.

Another aspect of the above embodiment, triggering of a modal change of an instrument is based on an observed event rather than a current condition. The modal change being maintained until a next trigger event is reached. Trigger events may be crossing into or out of the camera image boundary, reaching a particular angle of instrument relative to tissue, reversal of the angular crossing with a predetermined angle offset creating hysteresis or damping of triggered changes. Other offsets may be time, angle, distance as needed to prevent disabling an instrument or shifting modality during transient conditions often encountered during standard laparoscopic movements.

Another aspect of the invention, trigger events and offsets are adapted by cognitive networks employed in machine learning that algorithmically alter the triggers and trigger hysteresis. Such algorithms learn when a instrument is partially obstructed during active use rather than merely being out of view, i.e., when the distal extent of a bipolar vessel sealer or stapler jaw is locked onto tissue but jaw is obstructed by fluid adjacent anatomy in regular and appropriate use vs when unobservable.

In another aspect algorithmic control of the mode shifts and enablement may have a separate trigger threshold to prevent disruption of activation process like vessel sealing or stapler firing that might otherwise cause harm in failing to complete. The altered threshold would still prevent gross risk due to unobserved effects and may enable resumption of firing when trigger conditions are reversed.

In another aspect of the invention, control of an ultrasonic dissection instrument can be based on the orientation to the tissue being tissue in the jaw or adjacent the jaw tip or along the outside of the ultrasonically active jaw being an instrument with one active and one passive jaw. The energy mode or power being sealing for tissue in the jaw, increased power as the jaw is displaced from the local tissue surface, and an additional mode associated with tip cutting when only the distal end of the active jaw is in contact with tissue.

### BRIEF DESCRIPTION OF THE DRAWINGS

The present disclosure may be understood by reference to the accompanying drawings, when considered in conjunction with the subsequent, detailed description, in which:

FIG. 1 is a perspective view of an energy-based surgical system according to an embodiment of the present disclosure;

FIG. 2 is a perspective view of a surgical robotic system for use with the energy-based surgical system according to an embodiment of the present disclosure;

FIG. 3 is a front view of an energy generator of FIG. 1 according to an embodiment of the present disclosure;

FIG. 4 is a schematic diagram of the energy generator of FIG. 1 according to an embodiment of the present disclosure;

FIG. 5 is a flow chart of a method for controlling the energy-based surgical system of FIG. 1 according an embodiment of the present disclosure; and

FIGS. 6-10 are images of a surgical site corresponding to the flow chart of FIG. 5 processed by an endoscope controller of the energy-based surgical system of FIG. 1 according an embodiment of the present disclosure.

### DETAILED DESCRIPTION

Embodiments of the presently disclosed energy-based surgical system are described in detail with reference to the drawings, in which like reference numerals designate identical or corresponding elements in each of the several views. As used herein the term “distal” refers to the portion of the surgical instrument coupled thereto that is closer to the patient, while the term “proximal” refers to the portion that is farther from the patient.

The term “application” may include a computer program designed to perform functions, tasks, or activities for the benefit of a user. Application may refer to, for example, software running locally or remotely, as a standalone program or in a web browser, or other software which would be understood by one skilled in the art to be an application. An application may run on a controller, or on a user device, including, for example, a mobile device, an IOT device, a server system, or any programmable logic device.

In the following description, well-known functions or constructions are not described in detail to avoid obscuring the present disclosure in unnecessary detail. Those skilled in the art will understand that the present disclosure may be adapted for use with either an endoscopic instrument, a laparoscopic instrument, or an open instrument. It should also be appreciated that different electrical and mechanical connections and other considerations may apply to each particular type of instrument.

An energy generator according to the present disclosure may be used in monopolar and/or bipolar electrosurgical procedures, including, for example, cutting, coagulation, ablation, and vessel sealing procedures. The generator may include a plurality of outputs for interfacing with various ultrasonic and electrosurgical instruments (e.g., ultrasonic dissectors and hemostats, monopolar instruments, return electrode pads, bipolar electrosurgical forceps, footswitches, etc.). Further, the generator may include electronic circuitry configured to generate radio frequency energy specifically suited for powering ultrasonic instruments and electrosurgical devices operating in various electrosurgical modes (e.g., cut, blend, coagulate, division with hemostasis, fulgurate, spray, etc.) and procedures (e.g., monopolar, bipolar, vessel sealing).

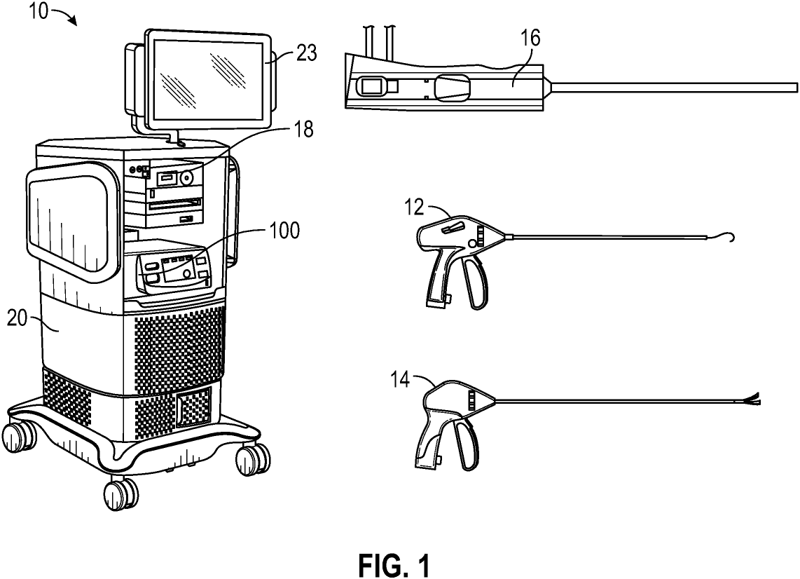

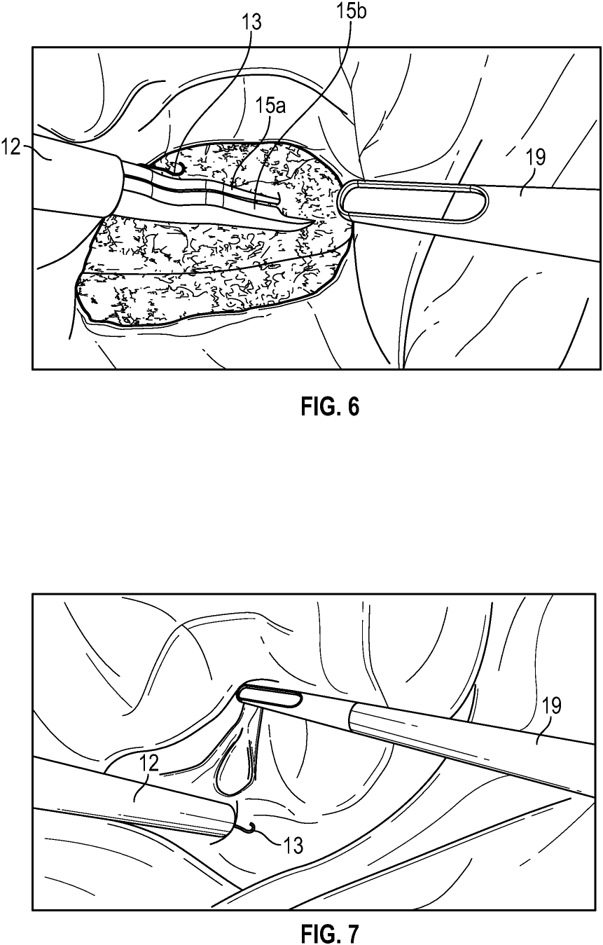

Referring to FIG. 1 an energy-based surgical system 10 is shown which may include a plurality of electrosurgical instruments, such as a first electrosurgical instrument 12, a second electrosurgical instrument 14, and an endoscope 16. The first and second electrosurgical instruments 12 and 14 may be monopolar, bipolar, or hybrid (monopolar and bipolar) such as LIGASURE™ instruments available from Medtronic, Minneapolis, MN. Instrument 12 may be a hybrid instrument and may include a pair of jaws 15a and 15b each having an electrode and used as bipolar forceps and an extendible monopolar electrode 13 (FIGS. 6 and 7). Instrument 14 may be a bipolar forceps instrument having a pair of jaws 17a and 17b each having an electrode (FIG. 10). In embodiments, the instrument 14 may be an ultrasonic instrument e.g., vessel sealer/dissector. The endoscope 16 may be an endoscopic camera that is coupled to an endoscope controller 18, which also provides light through a fiberoptic cable. The first and second electrosurgical instruments 12 and 14 are coupled to an energy generator 100. The endoscope controller 18 and the energy generator 100 are disposed in a control tower 20, which includes a display 23, which may be a touchscreen, and outputs the video feed from the endoscope 16 as well as various the graphical user interfaces (GUIs).

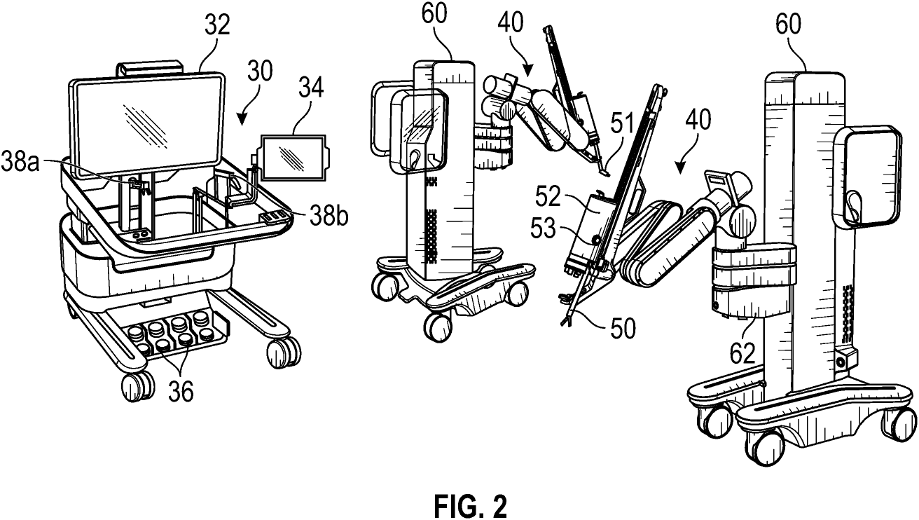

With reference to FIG. 2, the energy-based surgical system 10 may also be used as part of a surgical robotic system 11. The control tower 20 is connected to all of the components of the surgical robotic system 11 including a surgical console 30 and one or more robotic arms 40. Each of the robotic arms 40 includes a surgical instrument 50 (e.g., first and second electrosurgical instruments 12 and 14) removably coupled thereto. Each of the robotic arms 40 is also coupled to a movable cart 60.

The endoscope 16 is coupled to one of the robotic arms 40. The endoscope 16 is configured to capture video of the surgical site. The surgical console 30 includes a first display 32, which displays a video feed of the surgical site provided by camera 51 of the surgical instrument 50 disposed on the robotic arms 40, and a second interaction display 34, which displays a user interface for controlling the surgical robotic system 11. The first and second displays 32 and 34 are touchscreens allowing for displaying various graphical user inputs.

The surgical console 30 also includes a plurality of user interface devices, such as pedals 36 and a pair of handle controllers 38a and 38b which are used by a user to remotely control robotic arms 40. The surgical console further includes an armrest 33 used to support clinician’s arms while operating the handle controllers 38a and 38b.

The control tower 20 also acts as an interface between the surgical console 30 and one or more robotic arms 40. In particular, the control tower 20 is configured to control the robotic arms 40, such as to move the robotic arms 40 and the corresponding surgical instrument 50, based on a set of programmable instructions and/or input commands from the surgical console 30, in such a way that robotic arms 40 and the surgical instrument 50 execute a desired movement sequence in response to input from the foot pedals 36 and the handle controllers 38a and 38b.

Each of the control tower 20, the surgical console 30, and the robotic arm 40 includes a respective computer (not shown), which are interconnected to each other using any suitable communication network based on wired or wireless communication protocols. The term “network,” whether plural or singular, as used herein, denotes a data network, including, but not limited to, the Internet, Intranet, a wide area network, or a local area networks, and without limitation as to the full scope of the definition of communication networks as encompassed by the present disclosure. Suitable protocols include, but are not limited to, transmission control protocol/internet protocol (TCP/IP), datagram protocol/internet protocol (UDP/IP), and/or datagram congestion control protocol (DCCP). Wireless communication may be achieved via one or more wireless configurations, e.g., radio frequency - embedded millimeter wave transvers optical, Wi-Fi, Bluetooth (an open wireless protocol for exchanging data over short distances, using short length radio waves, from fixed and mobile devices, creating personal area networks (PANs), ZigBee® (a specification for a suite of high level communication protocols using small, low-power digital radios based on the IEEE 122.15.4-2003 standard for wireless personal area networks (WPANs)).

The computers of the robotic system 10 and the endoscope controller 18 may include any suitable processor (not shown) operably connected to a memory (not shown), which may include one or more of volatile, non-volatile, magnetic, optical, or electrical media, such as read-only memory (ROM), random access memory (RAM), electrically-erasable programmable ROM (EEPROM), non-volatile RAM (NVRAM), or flash memory. The processor may be any suitable processor (e.g., control circuit) adapted to perform the operations, calculations, and/or set of instructions described in the present disclosure including, but not limited to, a hardware processor, a field programmable gate array (FPGA), a digital signal processor (DSP), a central processing unit (CPU), a microprocessor, and combinations thereof. Those skilled in the art will appreciate that the processor may be substituted for by using any logic processor (e.g., control circuit) adapted to execute algorithms, calculations, and/or set of instructions described herein.

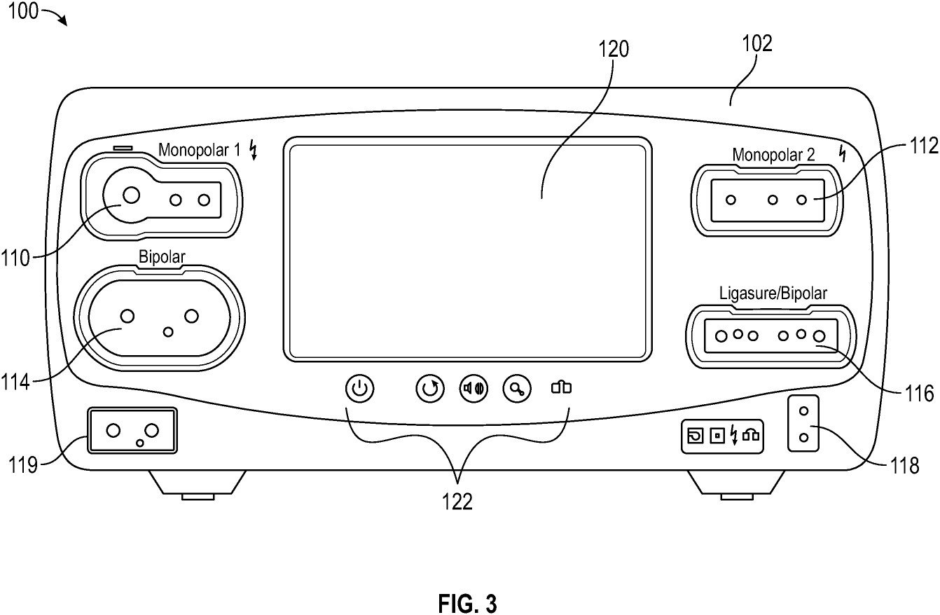

With reference to FIG. 3, a front face 102 of the generator 100 is shown. The generator 100 may include a plurality of ports 110, 112, 114, 116 to accommodate various types of electrosurgical instruments and a port 118 for coupling to a return electrode pad and a port 119 configured to couple to a footswitch. The ports 110 and 112 are configured to couple to the monopolar electrosurgical instruments (e.g., first electrosurgical instrument 12). The ports 114 and 116 are configured to couple to bipolar electrosurgical instruments (e.g., second electrosurgical instrument 14). The generator 100 includes a display 120 for providing the user with variety of output information (e.g., intensity settings, treatment complete indicators, etc.). The display 120 is a touchscreen configured to display a menu corresponding to each of the ports 110, 112, 114, 116 and the instrument coupled. The user also adjusts inputs by touching corresponding menu options. The generator 100 also includes suitable input controls 122 (e.g., buttons, activators, switches, touch screen, etc.) for controlling the generator 100.

The generator 100 is configured to operate in a variety of modes and is configured to output monopolar and/or bipolar waveforms corresponding to the selected mode. Each of the modes may be activated by the buttons disposed on the first and second electrosurgical instrument 12 and 14. Each of the modes operates based on a preprogrammed power curve that limits how much power is output by the generator 100 at varying impedance ranges of the load (e.g., tissue). Each of the power curves includes power, voltage and current control ranges that are defined by the user-selected intensity setting and the measured minimum impedance of the load.

The generator 100 may operate in the following monopolar modes, which include, but are not limited to, cut, blend, division with hemostasis, fulgurate and spray. The generator 100 may operate in the following bipolar modes, including bipolar cutting, bipolar coagulation, automatic bipolar which operates in response to sensing tissue contact, and various algorithm-controlled vessel sealing modes. The generator 100 may be configured to deliver energy required to power an ultrasonic transducer. Thereby enabling control and modulation of ultrasonic surgical instruments.

Each of the RF waveforms may be either monopolar or bipolar RF waveforms, each of which may be continuous or discontinuous and may have a carrier frequency from about 200 kHz to about 500 kHz. As used herein, continuous waveforms are waveforms that have a 100% duty cycle. In embodiments, continuous waveforms are used to impart a cutting effect on tissue. Conversely, discontinuous waveforms are waveforms that have a non-continuous duty cycle, e.g., below 100%. In embodiments, discontinuous waveforms are used to provide coagulation effects to tissue.

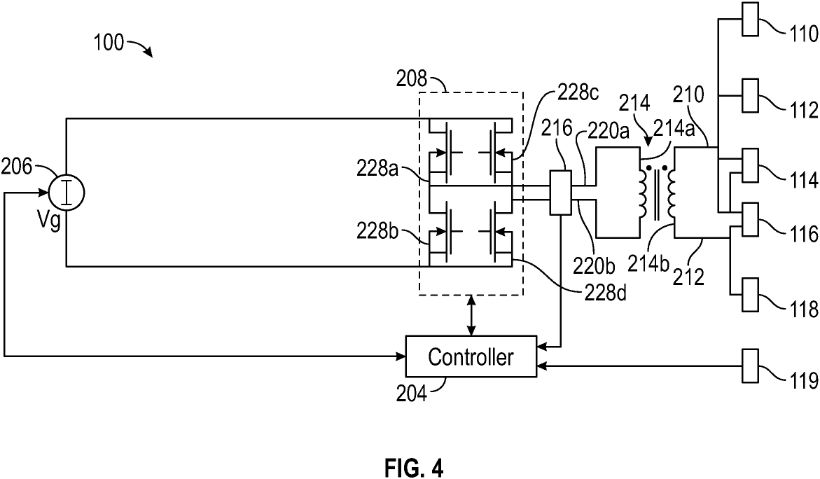

With reference to FIG. 4, the generator 100 includes a controller 204, a power supply 206, and a RF inverter 208. The power supply 206 may be high voltage, DC power supplies connected to a common AC source (e.g., line voltage) and provide high voltage, DC power to their respective RF inverter 208, which then convert DC power into a RF waveform through active terminal 210 and return terminal 212 corresponding to the selected mode.

The active terminal 210 and the return terminal 212 are coupled to the RF inverter 208 through an isolation transformer 214. The isolation transformer 214 includes a primary winding 214a coupled to the RF inverter 208 and a secondary winding 214b coupled to the active and return terminals 210 and 212.

Electrosurgical energy for energizing the monopolar electrosurgical instrument 20 is delivered through the ports 110 and 112, each of which is coupled to the active terminal 210. RF energy is returned through the return electrode pad coupled to the port 118, which in turn, is coupled to the return terminal 212. The secondary winding 214b of the isolation transformer 214 is coupled to the active and return terminals 210 and 212. RF energy for energizing a bipolar electrosurgical instrument is delivered through the ports 114 and 116, each of which is coupled to the active terminal 210 and the return terminal 212. The generator 100 may include a plurality of steering relays or other switching devices configured to couple the active terminal 210 and the return terminals 212 to various ports 110, 112, 114, 116, 118 based on the combination of the monopolar and bipolar electrosurgical instruments 20 and 30 being used.

The RF inverter 208 is configured to operate in a plurality of modes, during which the generator 100 outputs corresponding waveforms having specific duty cycles, peak voltages, crest factors, etc. It is envisioned that in other embodiments, the generator 100 may be based on other types of suitable power supply topologies. RF inverter 208 may be a resonant RF amplifier or non-resonant RF amplifier, as shown. A non-resonant RF amplifier, as used herein, denotes an amplifier lacking any tuning components, i.e., conductors, capacitors, etc., disposed between the RF inverter and the load, e.g., tissue.

The controller 204 may include a processor (not shown) operably connected to a memory (not shown) similar to the processors used in computers of the robotic system 10. The controller 204 is operably connected to the power supply 206 and/or RF inverter 208 allowing the processor to control the output of the RF inverter 208 of the generator 100 according to either open and/or closed control loop schemes. A closed loop control scheme is a feedback control loop, in which a plurality of sensors measures a variety of tissue and energy properties (e.g., tissue impedance, tissue temperature, output power, current and/or voltage, etc.), and provide feedback to the controller 204. The controller 204 then controls the power supply 206 and/or RF inverter 208, which adjust the DC and/or RF waveform, respectively.

The generator 100 according to the present disclosure may also include a plurality of sensors 216, each of which monitors output of the RF inverter 208 of the generator 100. The sensor 216 may be any suitable voltage, current, power, and impedance sensors. The sensors 216 are coupled to leads 220a and 220b of the RF inverter 208. The leads 220a and 220b couple the RF inverter 208 to the primary winding 214a of the transformer 214. Thus, the sensors 216 are configured to sense voltage, current, and other electrical properties of energy supplied to the active terminal 210 and the return terminal 212.

In further embodiments, the sensor 216 may be coupled to the power supply 206 and may be configured to sense properties of DC current supplied to the RF inverter 208. The controller 204 also receives input (e.g., activation) signals from the display 120, the input controls 122 of the generator 100 and/or the instruments 12 and 14. The controller 204 adjust power outputted by the generator 100 and/or perform other control functions thereon in response to the input signals.

The RF inverter 208 includes a plurality of switching elements 228a-228d, which are arranged in an H-bridge topology. In embodiments, RF inverter 208 may be configured according to any suitable topology including, but not limited to, half-bridge, full-bridge, push-pull, and the like. Suitable switching elements include voltage-controlled devices such as transistors, field-effect transistors (FETs), combinations thereof, and the like. In embodiments, the FETs may be formed from gallium nitride, aluminum nitride, boron nitride, silicon carbide, or any other suitable wide bandgap materials.

The controller 204 is in communication with the RF inverter 208, and in particular, with the switching elements 228a-228d. Controller 204 is configured to output control signals, which may be pulse-width modulated (“PWM”) signals, to switching elements 228a-228d. In particular, controller 204 is configured to modulate a control signal supplied to switching elements 228a-228d of the RF inverter 208. The control signal provides PWM signals that operate the RF inverter 208 at a selected carrier frequency. Additionally, controller 204 are configured to calculate power characteristics of output of the RF inverter 208 of the generator 100, and control the output of the generator 100 based at least in part on the measured power characteristics including, but not limited to, voltage, current, and power at the output of RF inverter 208.

The present disclosure provides for receiving and analyzing the video stream from the endoscope 16 at the endoscope controller 18 to extract contextual data from the video stream. The endoscope 16 may have a stereoscopic camera. In embodiments, the endoscope 16 may include an infrared source and a camera capable of capturing IR light and using the data to enhance the video feed. In further embodiments, thermal imaging, ultraviolet light image, and/or multi-spectral imaging devices may also be embedded in the endoscope 16. Furthermore, fiducials, tracers and contrast agents to enhance imaging and object detection may be used to enhance the video feed. The endoscope controller 18 may also incorporate additional imaging techniques such as depth mapping, laser speckle imaging for flow mapping, etc.

This contextual data may be, but is not limited to, detection of an instrument in the field of view (FOV) of the endoscope, detection of an implanted device in the FOV, detection of a specific tissue in the FOV, detection of a specific pathology in the FOV, detection of a specific tissue formations, such as tissue bundles, detection of aberrant instrument behavior such as arcing, detection of tissue reaction to therapy applied such as generation of steam and or smoke, tissue blanching, eschar formation, and the like.

Contextual data may be obtained using a computer vision algorithm derived from machine learning techniques, such as a deep neural network trained to recognize identity, position, orientation, operational state of instruments 12 and 14 in the field of view of the endoscope 16 and other contextual parameters described above. The deep learning neural network for classifying images (i.e., extract contextual data) may include a convolutional neural network (CNN) and/or a recurrent neural network. Generally, a deep learning neural network includes multiple hidden layers. The deep learning neural network may leverage one or more CNNs to classify one or more images, taken by the endoscope 16. In various methods, the one or more CNNs may have a different amount of classification from each other. For example, a first CNN may have a five-class CNN, and a second CNN may have a six-class CNN. The deep learning neural network may be executed on the endoscope controller 18.

The endoscope controller 18 is also coupled to the generator 100 and provides the contextual data to the generator 100. In embodiment, the controller 204 of the generator is configured to execute a control algorithm configured to perform one or more of the following automatic actions, including but not limited to, raising a visual warning on the display 23 and or display 120, raising a haptic warning through the instruments 12 and 14 and of on the control system for the relevant instrument, enabling or disabling an energy mode to prevent inadvertent activation out of FOV (beyond view, behind something, etc.), enabling or disabling an energy mode to prevent inadvertent activation near sensitive tissue structures, enabling or disabling an energy mode to prevent inadvertent activation near other instruments or implanted devices, switching energy modes, e.g., from monopolar to bipolar, RF to ultrasonic, isolated seal mode to bulk seal mode, modulating energy intensity, triggering sensors within the generator 100, the instruments 12 and 14, or another device, and triggering collection of data from the generator 100 or the endoscope 16.

During operation, there might be multiple triggering events. The endoscope controller 118 is configured to determine to change, disable or modify the RF energy output based on a first trigger event and disable or modify event is further modified or reversed based on a second trigger event offset from the first.

The control algorithm may be overridden by the clinician, including but not limited to, pressing a button or sequence of buttons, contextual based override where the output from the algorithm may be overridden by another algorithm, safety-based override to maintain proper function of instruments. In response to an override, the control algorithm may then take one or more of the following actions, including but not limited to, raising an audio and/or visual warning on the video feed source shown on the display 23 of the control tower 20 and/or GUI shown on the display 120 of the generator 100. Similarly, the controller 204 may provide haptic feedback to the instrument 12 or 14. In addition, an override may trigger collection of data relevant to the surrounding time period and event.

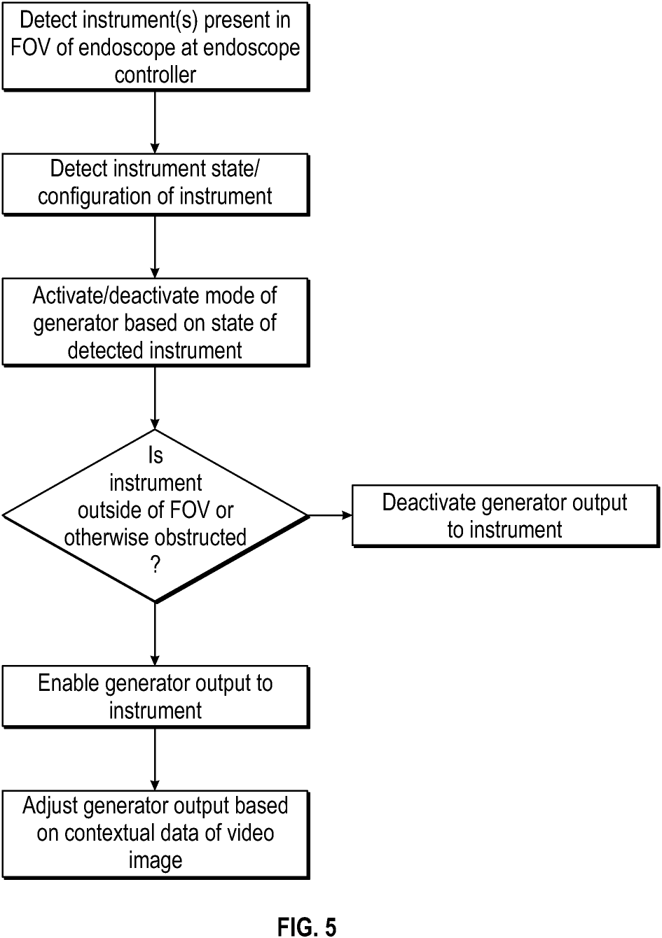

With reference to FIG. 5, a flow chart of operation of the endoscope controller 118 and the generator 100 includes detecting and identifying the instruments 12 and 14 as well as their operational status and location and controlling the generator 100 based on the same. The flow chart of FIG. 5 is described with respect to FIGS. 6-10 which show video frames captured by the endoscope 16.

The endoscope 16 continuously captures and transmits video data of the surgical site which is displayed on the display 23 and/or display 32, depending the system configuration being used. The endoscope controller 18 continuously processes the video data from the endoscope 16 and identifies the instruments 12 and 14 that are in the FOV of the endoscope 16. The endoscope controller 18 also detects the state of the instruments 12 and 14 such as whether jaws are open or closed, or whether a monopolar electrode 13 is retracted (FIG. 6) or extended (FIG. 7). FIGS. 6-8 show the instrument 12 and a grasper 19.

Based on the detected state of the instrument 12, the generator 100 is configured to enable or disable certain modes. Thus, when the generator 100 determines that the instrument 12 is in the bipolar configuration a corresponding bipolar mode is enabled in the generator 100, allowing for only energization of bipolar energy and disabling activation of monopolar energy. This is determined based on the position of the monopolar electrode 13. Thus, when the monopolar electrode 13 is retracted (FIG. 6), bipolar mode is enabled. Similarly, when the monopolar electrode 13 is extended (FIG. 7), the generator 100 determines that the instrument 12 is in the monopolar mode and enables monopolar mode while disabling bipolar mode. In addition, when the generator 100 determines that the instrument 14 is in the ultrasonic configuration a corresponding ultrasonic mode is enabled in the generator 100, allowing for only energizing an ultrasonic transducer. The generator 100 is also configured to switch between different energy modalities, e.g., from RF to ultrasonic, if the instrument 14 is capable of operating as an RF instrument and an ultrasonic instrument

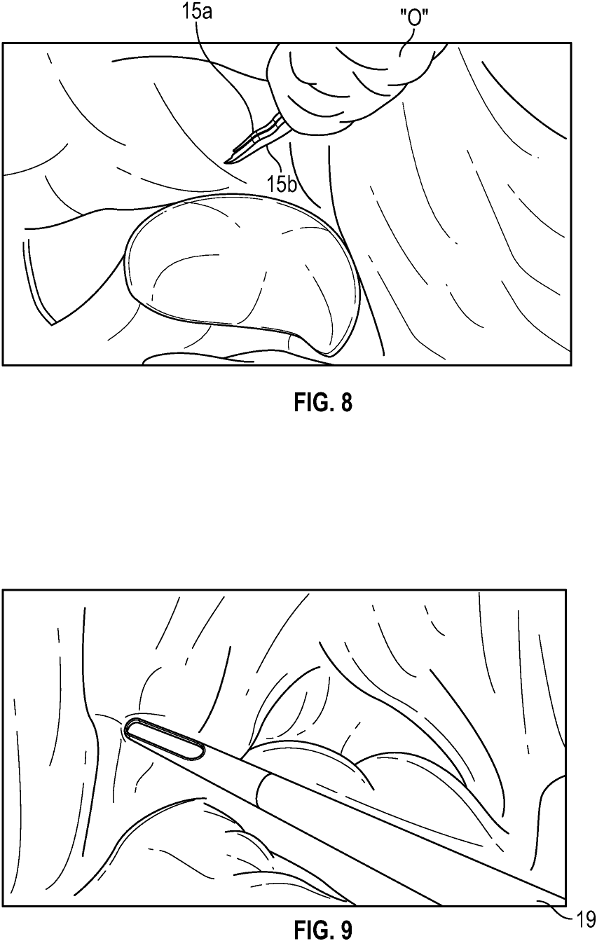

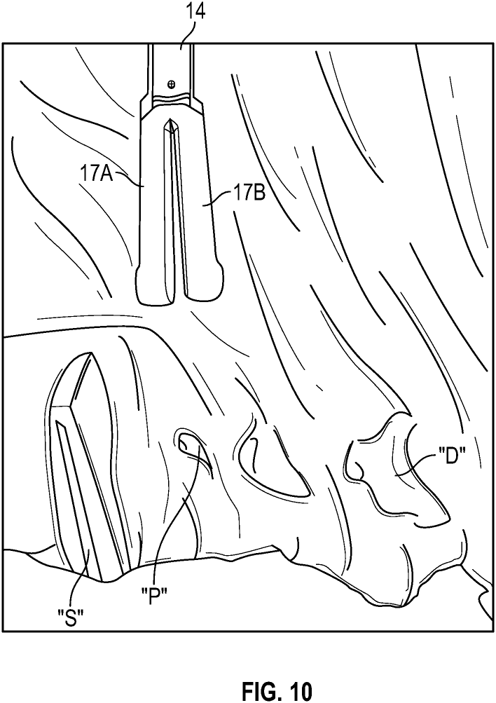

The generator 100 disables activation of the instrument 12 if the instrument 12 is obstructed by tissue (FIG. 8). As shown in FIG. 8, if tissue, e.g., an ovary “O”, is partially or wholly obscures the instrument 12, the endoscope controller 18 is incapable of determining the operational state of the instrument 12. As such, the endoscope controller 18 provides that contextual data to the generator 100, which prevents delivery of RF energy to the instrument 12, regardless of activation by the clinician. Similarly, if the instrument 12 is outside of FOV of the endoscope 16, the endoscope controller 18 determines that the instrument 12 is not visible (FIG. 9) and only the grasper 19 is visible. The generator 100 disables all energy (e.g., electrosurgical and/or ultrasonic) modes capable of energizing the instrument 12, such that the clinician cannot energize the instrument 12.

If the generator 100 determines that the instrument 12 is present in the FOV of the endoscope 16 without any obstructions, the generator 100 enables a corresponding energy mode. In embodiments, where the instrument includes only a single operational state, such as a monopolar or bipolar instrument, e.g., instrument 14, the generator 100 simply enables the corresponding electrosurgical mode, e.g., monopolar or bipolar, rather than making a selection of the corresponding mode based on the state of the instrument, e.g., instrument 12.

With reference to FIG. 10, the endoscope controller 18 is also configured to analyze video data to extract contextual data pertaining to the tissue effect imparted by application of RF energy. The endoscope controller 18 is also configured to determine whether the tissue being operated on is within dimensional thresholds to avoid including too much tissue between jaws 17a and 17b of instrument 14. The endoscope controller 18 is also configured to determine placement of the tissue between the jaws 17a and 17b, such as whether the tissue is centered or disposed toward more proximal “P” or distal “D” ends of the jaws 17a and 17b. The endoscope controller 18 is further configured to determine the angle between the jaws 17a and 17b. The angle and tissue placement are used by the generator 100 to control energy delivery to the instrument 14 by changing the intensity of the selected mode.

In embodiments, the endoscope controller 18 is also configured to determine changes in tissue, such as detecting bleeding and modifying and/or selecting a different mode to apply sufficient RF energy to seal the bleeding vessel. Unlike conventional generators which utilize impedance and other energy properties to correlate increases in impedance with bleeding, the present disclosure provides for visual confirmation and automatic detection and control of RF energy based on video data. In response to automatic changes in mode selection or intensity adjustment, the generator 100 is configured to provide haptic feedback to the instrument 12 or 14. The endoscope controller 18 is further configured to monitor tissue treatment progress, such as determining thermal spread “S” during vessel sealing, such that if the spread is beyond the jaws 17a and 17b, the generator 100 is configured to stop application of RF energy or lower intensity of the RF energy. In addition, the endoscope controller 18 is also configured to detect critical tissue structures, e.g., ovaries “O” (FIG. 8), and the generator 100 is configured to adjust RF output based on detection of such critical structures and proximity of the instrument 14 to the critical structures to avoid injury thereto. Adjustments may include changing intensity of the preset intensity of the mode, stopping energy entirely, or directing energy in one direction away from the critical structure.

While several embodiments of the disclosure have been shown in the drawings and/or described herein, it is not intended that the disclosure be limited thereto, as it is intended that the disclosure be as broad in scope as the art will allow and that the specification be read likewise. Therefore, the above description should not be construed as limiting, but merely as exemplifications of particular embodiments. Those skilled in the art will envision other modifications within the scope of the claims appended hereto.

What is claimed is:

1. An energy-based surgical system comprising:
at least one energy instrument;
an endoscope configured to capture video data of a surgical site including the at least one energy instrument;
an endoscope controller configured to process the video data to determine contextual data pertaining to one of the surgical site or the at least one energy instrument; and
an energy generator coupled to the at least one energy instrument, the energy generator configured to generate an energy output to the at least one energy instrument and to control the energy output based on the contextual data.

2. The energy-based surgical system according to claim 1, wherein the contextual data includes at least one of type, operational state, position, or orientation of the at least one energy instrument.

3. The energy-based surgical system according to claim 1, wherein the at least one energy instrument is an electrosurgical instrument operable in a plurality of configurations.

4. The energy-based surgical system according to claim 3, wherein the energy generator is further configured to select an electrosurgical mode based on a configuration of the at least one energy instrument.

5. The energy-based surgical system according to claim 1, wherein the endoscope controller is further configured to determine whether the at least one energy instrument is obstructed or outside of field of vision of the endoscope.

6. The energy-based surgical system according to claim 5, wherein the energy generator is further configured to disable the energy output based on determination whether the at least one energy instrument is at least one obstructed or outside of field of vision of the endoscope.

7. The energy-based surgical system according to claim 1, wherein the at least one energy instrument is an electrosurgical bipolar forceps configured to form a tissue seal.

8. The energy-based surgical system according to claim 7, wherein the endoscope controller is further configured to determine at least one parameter of the tissue seal.

9. The energy-based surgical system according to claim 8, wherein the energy generator is configured to adjust the energy output based on the at least one parameter of the tissue seal.

10. The energy-based surgical system according to claim 1, wherein the at least one energy instrument is an ultrasonic dissection instrument.

11. The energy-based surgical system according to claim 10, wherein the energy generator is further configured to adjust the energy output or vibration duration in response to an orientation of the ultrasonic dissection instrument relative to tissue.

12. The energy-based surgical system according to claim 1, wherein the energy output is at least one radio frequency energy, ultrasound energy, laser energy, or thermal energy.

13. An energy-based surgical system comprising:
a plurality of electrosurgical instruments;
an endoscope configured to capture video data of a surgical site including the electrosurgical instruments;
an endoscope controller configured to process the video data to determine contextual data pertaining to one of the surgical site or the electrosurgical instruments and to identify each of the plurality of electrosurgical instruments; and
an energy generator coupled to the electrosurgical instruments, the energy generator configured to provide radio frequency (RF) energy output to the electrosurgical instruments and to control the RF energy output based on the contextual data.

14. The energy-based surgical system according to claim 13, wherein the contextual data includes at least one of type, operational state, position, or orientation of each of the plurality of electrosurgical instruments.

15. The energy-based surgical system according to claim 13, wherein the endoscope controller is further configured to determine whether one of the plurality of electrosurgical instruments is obstructed or outside of field of vision of the endoscope and the energy generator is further configured to disable the RF energy output based on determination whether one of the plurality of electrosurgical instruments is at least one obstructed or outside of field of vision of the endoscope.

16. A method for controlling an energy generator, the method includes:
capturing video data of a surgical site and at least one electrosurgical instrument through an endoscope;
processing the video data at an endoscope controller to determine contextual data pertaining to one of the surgical site or the at least one electrosurgical instrument; and
outputting radio frequency (RF) energy to the at least one electrosurgical instrument from an energy generator; and
controlling RF energy based on the contextual data.

17. The method according to claim 16, further comprising operating the at least one electrosurgical instrument in one of a plurality of configurations.

18. The method according to claim 17, further comprising selecting at the energy generator an electrosurgical mode based on a configuration of the at least one electrosurgical instrument.

19. The method according to claim 16, further comprising determining whether the at least one electrosurgical instrument is obstructed or outside of field of vision of the endoscope.

20. The method according to claim 19, further comprising disabling the RF energy output based on determination whether the at least one electrosurgical instrument is at least one obstructed or outside of field of vision of the endoscope.

21. The method according to claim 16, wherein the at least one electrosurgical instrument is a bipolar forceps configured to form a tissue seal.

22. The method according to claim 21, further comprising determining at least one parameter of the tissue seal.

23. The method according to claim 22, further comprising adjusting the RF energy output based on the at least one parameter of the tissue seal.
